(19) United States
(12) Patent Application Publication
(10) Pub. No.: US 2017/0209655 A1
(43) Pub. Date: Jul. 27, 2017

(54) DRY POWDER MEDICAMENT DE-AGGLOMERATOR

(71) Applicant: MICRODOSE THERAPEUTX, INC., Ewing, NJ (US)

(72) Inventor: Mark Steven Morrison, Basking Ridge, NJ (US)

(21) Appl. No.: 15/325,003

(22) PCT Filed: Jul. 21, 2015
(86) PCT No.: PCT/US15/41403

§ 371 Date: Jan. 9, 2017

Related U.S. Application Data
(60) Provisional application No. 62/028,169, filed on Jul. 23, 2014.

(51) Int. Cl. A61M 15/00

(52) U.S. Cl. CPC A61M 15/0008; A61M 15/001; A61M 15/0035; A61M 15/0045; A61M 2202/064; A61M 2206/16; A61M 2206/14

(57) ABSTRACT

A de-agglomerator comprises: a medicament chamber comprising an inlet and outlet and having a cross-section with a substantially circular inner perimeter; a conduit in pneumatic communication with said inlet; and a blower arranged to direct airflow at dry powder medication through said conduit. Another de-agglomerator comprises: a piezoelectric transducer; a pumping chamber: coupled to said transducer through a diaphragm, and having a outlet hole: in a wall opposing the transducer, in line with the transducer axis, and arranged to be in pneumatic communication with a flow channel of said inhaler through which a user can inhale; and a housing surrounding said pumping chamber, comprising a housing inlet in a wall facing the transducer; a housing outlet in a wall facing said outlet hole and in line with the outlet hole; and being spaced from the pumping chamber by an airflow channel in pneumatic communication with the housing inlet and outlet.

The present disclosure relates generally to the field of delivery of pharmaceuticals. In particular, devices and methods for delivery of dry powder medicaments by inhalation therapy are considered, including de-agglomerators for dry powder inhalers, methods of de-agglomerating dry powder medicament in inhalers, a capsule arranged for use in a de-agglomerator, a blister pack comprising an array of such capsules and inhalers comprising such de-agglomerators.

Certain diseases of the respiratory tract are known to respond to treatment by the direct application of therapeutic agents. As these agents are most readily available in dry powdered form, their application is most conveniently accomplished by inhaling the powdered material through the nose or mouth. This powdered form results in the better utilization of the medication in that the drug is deposited exactly at the site desired and where its action may be required; hence, very minute doses of the drug are often equally as efficacious as larger doses administered by other means, with a consequent marked reduction in the incidence of undesired side effects and medication cost. Alternatively, the drug in powdered form may be used for treatment of diseases other than those of the respiratory system. When the drug is deposited on the very large surface areas of the lungs, it may be very rapidly absorbed into the blood stream; hence, this method of application may take the place of administration by injection, tablet, or other conventional means.

Bioavailability is generally considered to be optimised when the drug particles delivered to the respiratory tract are between about 1 and 5 microns in size. When the drug particles need to be in this size range the dry powder delivery system needs to address a number of issues, including the following.
Small size particles develop an electrostatic charge on themselves during manufacturing and storage. This causes the particles to agglomerate or aggregate, resulting in clusters of particles which have an effective size greater than 5 microns. The probability of these large clusters making it to the deep lungs then decreases. This in turn results in a lower percentage of the drug being available to the patient for absorption.
The amount of active drug that needs to be delivered to the patient may be of the order of tens of micrograms. Since current powder filling equipment cannot effectively deliver aliquots of drugs in microgram quantities with acceptable accuracy, the standard practice is to mix the active drug with a filler or bulking agent (sometimes known as a carrier) such as lactose. This additive also makes the drug “easy to flow”. Carrier particles are often larger than the drug particles in size. The ability of the dry powder inhaler to separate drug from the carrier is an important performance parameter in the effectiveness of the design.
Active drug particles with sizes greater than 5 microns tend to be deposited either in the mouth or throat. This introduces another level of uncertainty since the bioavailability and absorption of the drug in these locations is different from the lungs. Dry powder inhalers need to minimize the drug deposited in these locations to reduce the uncertainty associated with the bioavailability of the drug.

Prior art dry powder inhalers (DPIs) usually have a means for introducing the drug (active drug plus carrier) into a high velocity air stream. The high velocity air-stream is used as the primary mechanism for breaking up the cluster of micronized particles or separating the drug particles from the carrier. Several inhalation devices useful for dispensing this powder form of medication are known in the prior art. For example, in U.S. Pat. Nos. 3,507,277; 3,518,992; 3,635,219; 3,795,244; and 3,807,400, inhalation devices are disclosed having means for piercing or removing the top of a capsule containing a powdered medication, which upon inhalation is drawn out of the pierced or topped capsule and into the user's mouth. Several of these patents disclose propeller means, which upon inhalation aid in dispensing the powder out of the capsule, so that it is not necessary to rely solely on inhalation to suction powder from the capsule.

Prior art devices such as those described above have a number of disadvantages which makes them less than desirable for the delivery of dry powder to the lungs, including the following.
The performance of the prior art inhalers depends on the flow rate generated by the user. Lower flow rate does not result in the powder being totally de-aggregated and hence adversely affects the dose delivered to the patient.
Inconsistency in the bioavailability of the drugs from dose-to-dose because of lack of consistency in the de-aggregation process (due to sensitivity to user-generated flow rate).
Large energy requirements for driving the electromechanical based inhalers which increases the size of the devices making them unsuitable for portable use.
Loss of medication from opened or topped capsules.
Deterioration of medication in open or topped capsule due to exposure to oxygen and/or moisture.

The foregoing discussion of the prior art derives in part from U.S. Pat. No. 7,318,434, which describes a dry powder inhaler which employs synthetic jetting technology to aerosolize drug powder from a blister pack or the like. It is known that if one uses a chamber bounded on one end by an acoustic wave generating device and bounded on the other end by a rigid wall with a small orifice, that when acoustic waves are emitted at high enough frequency and amplitude from the generator, a jet of air that emanates from the orifice outward from the chamber can be produced. The jet, or so-called “synthetic jet”, is comprised of a train of air puffs that are formed at the orifice at the generator's frequency. However, as described in the aforesaid '434 patent, the use of a synthetic jet to de-aggregate and eject a dry-powder material from a blister pack or the like provides advantages over prior art dry powder inhalers.

More particularly, the aforesaid '434 patent provides a dry powder inhaler having a first chamber for and holding a dry powder, and a second chamber connected to the first chamber via a passageway for receiving an aerosolized form of the dry powder from the first chamber and for delivering the aerosolized dry powder to a user. A vibrator is coupled to the dry powder in the first chamber. Since jetting efficiency falls off as the aspect ratio (length to cross-section or diameter) of the passageway increases, in order to create a synthetic jet the passageway connecting the first chamber to the second chamber preferably, but not necessarily, has an aspect ratio equal to at least about one, and the vibrator is energised and coupled to the first chamber so that the distance the gas moves back and forth in the passageway is at least about twice the cross-section or diameter of the passageway.

In a first embodiment of the aforesaid '434 patent, the first chamber is formed in the shape of a cylinder or blister with a vibratory element either forming one wall of the chamber, or the vibratory element being formed apart from the chamber and coupled to the blister.

In a second embodiment of the aforesaid '434 patent the first chamber is formed in the shape of a horn, with a vibratory element either forming one wall of the chamber, or the vibratory element being coupled to a wall of the chamber via a column of gas.

In a third embodiment of the aforesaid '434 patent the first chamber is formed in the shape of a horn, and a standing wave resonator is coupled to a wall of the chamber.

See also U.S. Pat. Nos. 7,334,577; 7,779,837 and 8,322,338.

The blister implementation described in the aforementioned patents bears some resemblance to an inverted kettle drum, whereby a piezoelectric transducer (piezo) applies acoustic energy to the open end of the chamber (i.e. drum). Small holes at the closed end provide an escape path for drug loaded in the chamber. When driven at the right frequency, as governed by dimensions of both the piezo and the chamber, a unique standing wave pattern is created that, owing to the unique shape of the chamber, results in the formation of pressure anti-nodes at both ends, with a pressure node in between.

The pressure anti-node nearest the closed end of the chamber works in concert with the small holes at that end to create synthetic jets which expel drug from the chamber. Synthetic jetting is the phenomenon by which air passing rapidly through an opening develops vortices that move away from the opening. The same thing happens in the opposite direction, at different times, such that the net air mass flow is zero. These ‘internal vortices’ (or jets) assist with mixing of drug powder within the chamber. However, the vortices leaving the chamber carry with them powdered drug, which leaves the chamber and does not return. These are the particles available for patient inhalation.

According to a first aspect, there is provided a method for delivering dry powder medication to a patient for inhalation therapy, said method comprising: providing a dry powder inhaler having a substantially circular chamber containing said powder medication; directing acoustic waves at the dry powder medication contained in the chamber, wherein the acoustic waves cause the dry powder to swirl around an inner circumference of the chamber such that agglomeration of the dry powder is reduced; and ejecting de-agglomerated particles from the chamber.

The acoustic waves could be produced by a piezoelectric transducer.

The acoustic waves could be directed along an axis tangential to the inner circumference of the circular chamber.

The circular chamber could have internal baffles.

According to a second aspect, there is provided a device for delivering dry powder medication to a patient for inhalation therapy, said device comprising: a dry powder inhaler having circular chamber containing said powder medication; an acoustic wave generator arranged to direct acoustic waves at the dry powder medication contained in the chamber, wherein the acoustic waves cause the dry powder to swirl around an inner circumference of the chamber such that agglomeration of the dry powder is reduced; and an outlet in the chamber for ejecting de-agglomerated particles from the chamber.

The acoustic wave generator could comprise a piezoelectric transducer.

The device could be arranged such that the acoustic waves are directed along an axis tangential to the inner circumference of the circular chamber.

The circular chamber could have internal baffles.

According to a third aspect, there is provided a capsule comprising a substantially circular chamber and arranged for use in the device of the second aspect.

According to a fourth aspect, there is provided a blister pack comprising an array of substantially circular chambers arranged such that each chamber can be used as a chamber in the device of the second aspect.

According to a fifth aspect, there is provided a de-agglomerator for a dry powder inhaler, said de-agglomerator comprising: a medicament chamber for housing dry powder medication, said medicament chamber comprising an inlet and an outlet and having a cross-section with a substantially circular inner perimeter; a conduit in pneumatic communication with said inlet; and a blower arranged to direct airflow at said dry powder medication through said conduit such that the medicament swirls around at least a part of said perimeter.

The conduit could be tangential to the perimeter.

The blower could comprise a piezoelectric transducer.

The medicament chamber could comprise one or more internal baffles.

Said blower could be an acoustic streaming blower.

Said blower could comprise a tube closed at one end by said transducer, an open end of said tube being in pneumatic communication with the conduit.

Said transducer could be, or could be coupled to, a focussing dish, concave with respect to the airflow direction.

Said blower could be a synthetic jet or pumping blower.

Said blower could comprise a pumping chamber coupled to said transducer and having at least one outlet hole in pneumatic communication with the conduit.

Said outlet hole could be in a wall of said pumping chamber opposing the transducer.

The transducer could be coupled to the pumping chamber through a diaphragm.

The outlet hole could be in line with the transducer axis.

The pumping chamber could be surrounded by a housing, said housing: comprising a housing inlet in a wall facing the transducer; comprising a housing outlet in a wall facing the outlet hole and in line with the outlet hole; and being spaced from the pumping chamber by an airflow channel in pneumatic communication with the housing inlet and the housing outlet.

According to a sixth aspect, there is provided a method of de-agglomerating dry powder medicament in an inhaler, said method comprising, using a blower, directing airflow at said medicament through a conduit such that the medicament swirls around at least a part of a substantially circular inner perimeter of a medicament chamber housing the medicament, said medicament chamber comprising an inlet in pneumatic communication with said conduit and an outlet.

According to a seventh aspect, there is provided a de-agglomerator for a dry powder inhaler, said de-agglomerator comprising: a piezoelectric transducer; a pumping chamber: coupled to said transducer through a diaphragm, and having a outlet hole: in a wall opposing the transducer, in line with the transducer axis, and arranged to be in pneumatic communication with a flow channel of said inhaler through which a user can inhale; and a housing surrounding said pumping chamber, said housing: comprising a housing inlet in a wall facing the transducer; comprising a housing outlet in a wall facing said outlet hole and in line with the outlet hole; and being spaced from the pumping chamber by an airflow channel in pneumatic communication with the housing inlet and the housing outlet.

Said pumping chamber could be arranged for housing dry powder medicament.

The de-agglomerator could further comprise a dosing chamber for housing dry powder medication, said dosing chamber comprising: an opening configured to receive an open dry powder medicament blister such that dry powder medicament can pass into the dosing chamber from said blister; an outlet in pneumatic communication with said flow channel; and an inlet in pneumatic communication with said outlet hole, configured to direct airflow from the outlet hole at said opening.

The de-agglomerator could further comprise: a medicament chamber for housing dry powder medication, said medicament chamber comprising an inlet and an outlet in pneumatic communication with said flow channel and having a cross-section with a substantially circular inner perimeter; and a conduit in pneumatic communication with said inlet; wherein the outlet hole is arranged to direct airflow at said dry powder medication through said conduit such that the medicament swirls around at least a part of said perimeter.

The conduit could be tangential to the perimeter.

The medicament chamber could comprise one or more internal baffles.

According to an eighth aspect, there is provided a capsule comprising the medicament chamber and arranged for use in the de-agglomerator of the fifth or seventh aspects.

According to a ninth aspect, there is provided a blister pack comprising an array of the capsules of the eighth aspect.

According to a tenth aspect, there is provided a method of de-agglomerating dry powder medicament in an inhaler, said method comprising: providing a pumping chamber with said dry powder medicament; and supplying an alternating current to a piezoelectric transducer coupled to said pumping chamber through a diaphragm such that the volume of the pumping chamber varies with the supplied voltage, the pumping chamber: having a outlet hole: in a wall opposing the transducer, in line with the transducer axis, and arranged to be in pneumatic communication with a flow channel of said inhaler through which a user can inhale, the pumping chamber being surrounded by a housing, said housing: comprising a housing inlet in a wall facing the transducer; comprising a housing outlet in a wall facing said outlet hole and in line with the outlet hole; and being spaced from the pumping chamber by an airflow channel in pneumatic communication with the housing inlet and the housing outlet.

According to an eleventh aspect, there is provided a method of de-agglomerating dry powder medicament in an inhaler, said method comprising: providing a medicament chamber with said dry powder medicament, said medicament chamber: comprising an inlet and an outlet, being in pneumatic communication, via said outlet, with a flow channel of said inhaler through which a user can inhale, and having a cross-section with a substantially circular inner perimeter; and supplying an alternating current to a piezoelectric transducer coupled to a pumping chamber through a diaphragm such that the volume of the pumping chamber varies with the supplied voltage, the pumping chamber: having a outlet hole: in a wall opposing the transducer, in line with the transducer axis, and arranged to direct airflow at said dry powder medication through a conduit in pneumatic communication with said inlet such that the medicament swirls around at least a part of said perimeter, the pumping chamber being surrounded by a housing, said housing: comprising a housing inlet in a wall facing the transducer; comprising a housing outlet in a wall facing said outlet hole and in line with the outlet hole; and being spaced from the pumping chamber by an airflow channel in pneumatic communication with the housing inlet and the housing outlet.

According to a twelfth aspect, there is provided an inhaler comprising the de-agglomerator of the fifth or seventh aspects.

Aspects of the present invention will now be described by way of example with reference to the accompanying figures. In the figures:

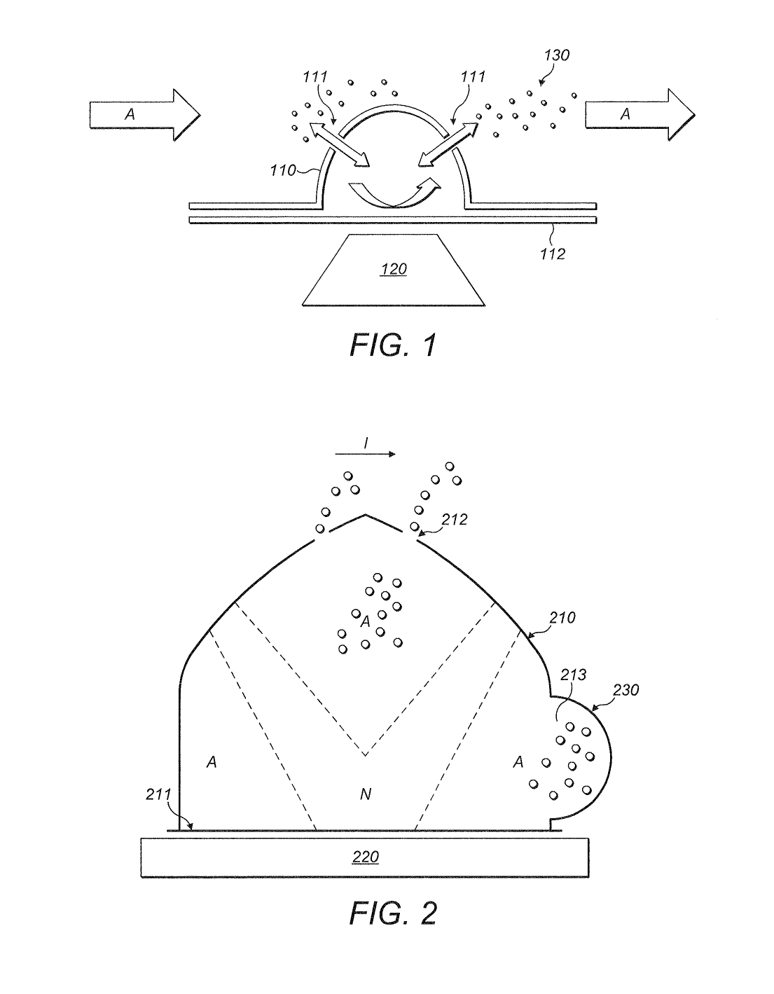

FIG. 1 shows a drug blister;

FIG. 2 shows a dosing chamber;

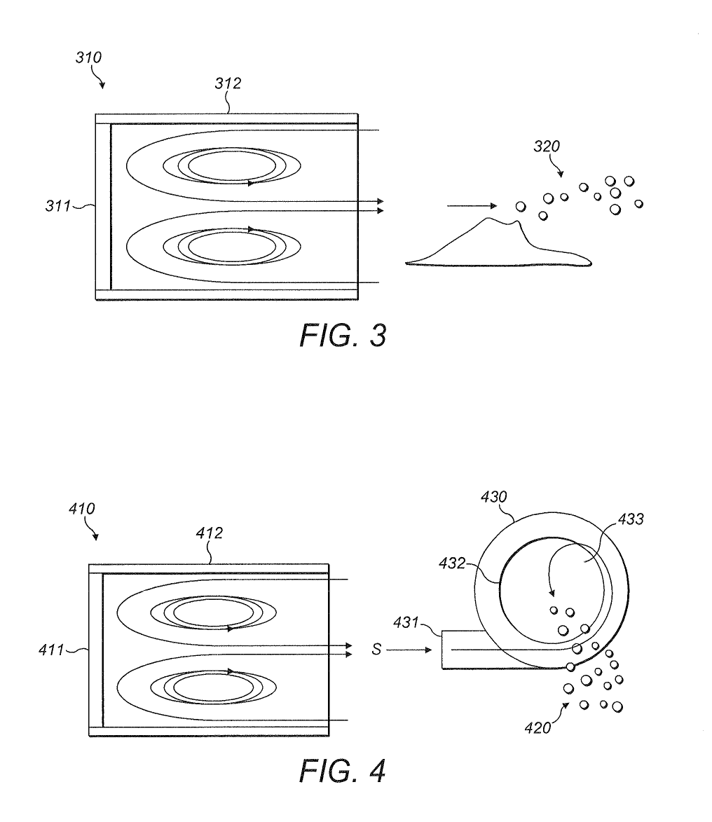

FIG. 3 illustrates Eckart streaming;

FIG. 4 shows a blower employed in an example de-agglomerator arrangement;

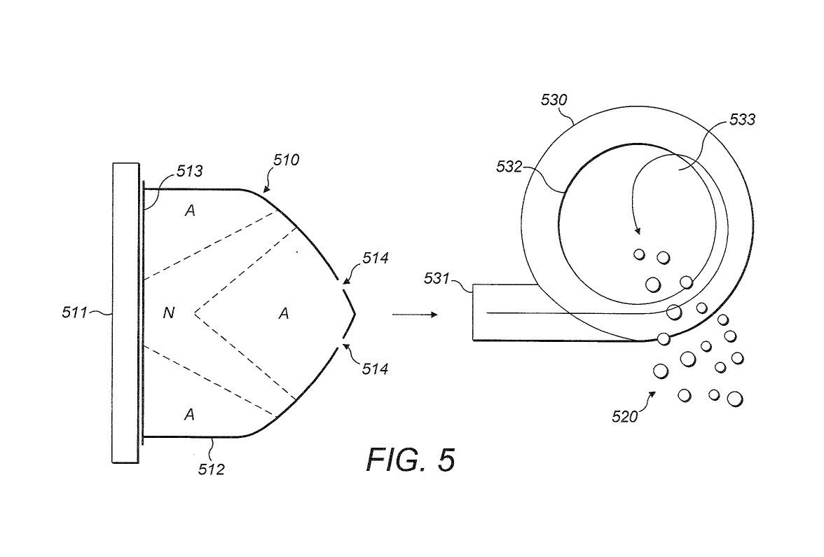

FIG. 5 shows an example arrangement comprising an alternative blower;

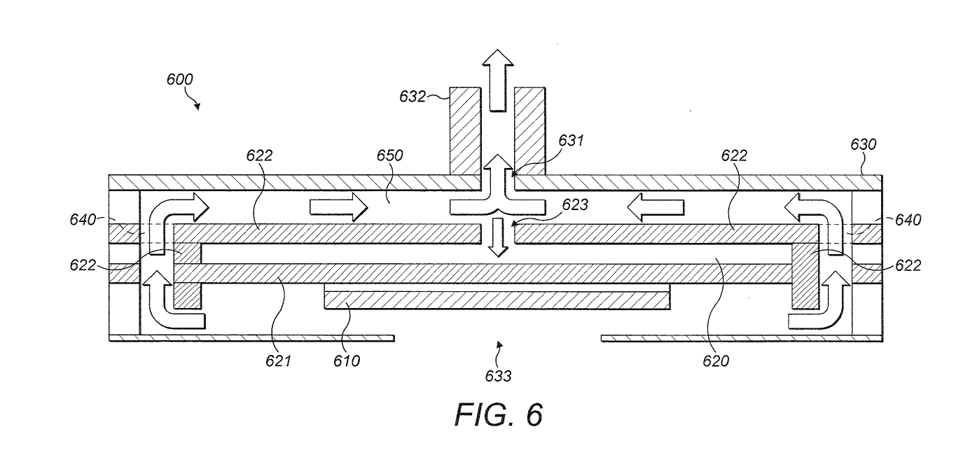

FIG. 6 illustrates a further type of blower;

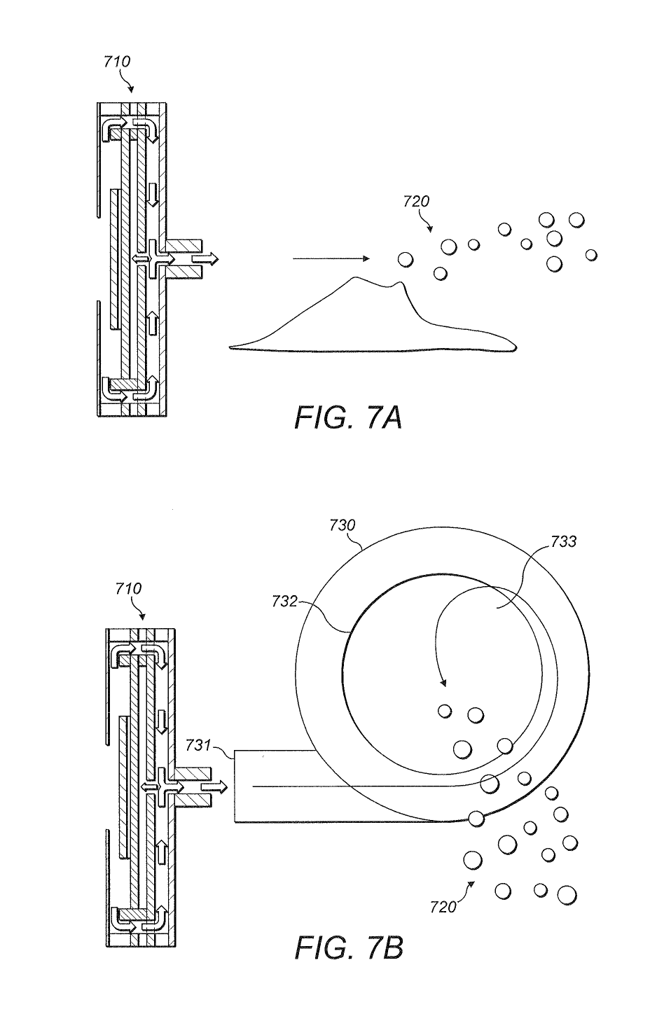

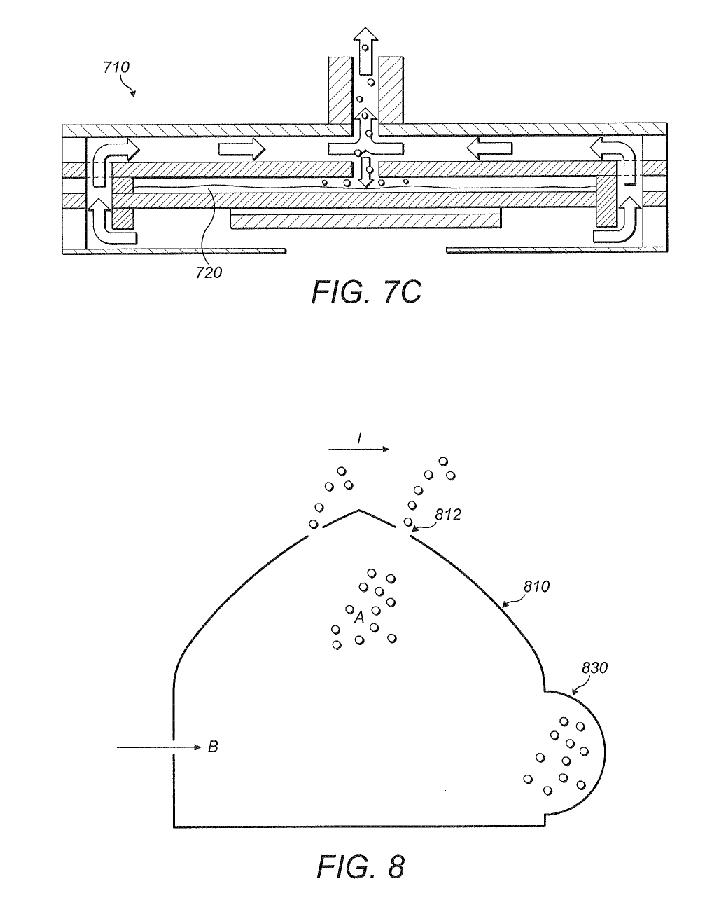

FIGS. 7A to 7C show the blower of FIG. 6 employed in various example arrangements; and

FIG. 8 illustrates another example de-agglomerator.

The following description is presented to enable any person skilled in the art to make and use the system, and is provided in the context of a particular application. Various modifications to the disclosed embodiments will be readily apparent to those skilled in the art.

With the apparatuses and methods described herein it is possible to deliver a dry powder drug for inhalation in a simple and reliable way with a device which is compact and low cost yet is capable of effective delivery with low power requirements.

Referring to FIG. 1, a known design uses a special dome shaped drug blister 110 as the chamber. This requires a special piercing tool to create the outlet holes 111 just prior to use. In this case, the piezo 120 is placed in contact with the lidding material 112 of the sealed blister, vibrating the bottom of the blister and causing direct agitation of the drug powder within. In this capacity, the piezo both creates acoustic waves that result in synthetic jetting and de-agglomerates the drug resting on the lid material by direct vibration. The drug 130 is then picked up in the inspiratory airflow indicted by arrows A.

More recently, an alternative has been designed, and is a drug delivery system comprising a dose chamber coupled to a vibrating device as described in U.S. Pat. No. 8,991,390. In an embodiment described in the '390 patent, an inhaler is provided with a combined reservoir and dosing chamber configured to receive multiple doses of a pharmaceutical material. As before, the dosing chamber is coupled to a vibration device for aerosolising the pharmaceutical, and delivering aerosolised pharmaceuticals to the patient.

As illustrated in FIG. 2, the hard dosing chamber described in the '390 patent has been modified to include a thin membrane 211 that serves to both seal off the dosing chamber 210 as well as couple the chamber to the vibrating device (e.g. piezo) 220. FIG. 2 shows the locations of pressure antinodes A and pressure node N in this arrangement. The direction of inspiratory airflow is indicated by arrow I.

As can be seen, a thin plastic film now covers the open end, through which the piezo applies acoustic energy. Small outlet holes 212 are moulded into the chamber, replacing those created in the design of FIG. 1 by way of piercing. In this case, the drug blister 230 has been relocated to the side of the chamber, where its contents are delivered to the chamber through a small opening 213 in the chamber wall, the lidding material having been previously peeled back. In this position, the opening of the blister is placed in close proximity to a pressure anti node (A) on the outer circumference of the chamber. The transport of drug from the blister to the chamber is thought to be facilitated by pressure variations at the antinode as well as direct vibration of the piezo coupled into the blister by way of the surrounding structure, which is in communication with the piezo.

The arrangements of FIGS. 1 and 2 employ synthetic jetting to transport powdered drug to the patient for inhalation. However, something similar can also be done using acoustic streaming, the phenomenon by which sound travelling through a medium imparts momentum to that medium, causing it to move. Of interest to this disclosure is the Eckart streaming which can be demonstrated using a common 40 kHz piezo transducer as illustrated in FIG. 3. As one example, if such a transducer 311 is driven at sufficiently high amplitude into an open ended tube 312, it is possible to displace powders 320 by the air flow so generated. The effect is not particularly strong, and may not be sufficient to de-agglomerate all drug pellets, but it is adequate to aerosolise already de-agglomerated fine powders. This can be thought of as a “blower” 310 capable of aerosolising drug for entrainment within the patient inhalation flow.

FIG. 4 shows such a blower employed in a de-agglomerator arrangement. Blower 410 has a piezoelectric component (piezo) 411 which can agitate a drug 420. This is done by the piezo 411 creating a flow of air along an axis A, by way of acoustic streaming, which passes over the drug 420 to de-agglomerate the drug and move particles of it.

To use acoustic streaming for dry powder nebulization, one needs to direct the sound axis of the piezo in the direction of the drug load. The specially designed container 412 enhances the effects by directing the sound. The effect can be enhanced even further by using piezo transducers that include certain focusing features. For example, the transducer itself could be a concave (e.g. parabolic or hemispherical) dish, or could be coupled to such a dish e.g. via mounting with a compliant adhesive such as silicone.

In order to improve de-agglomeration of the drug powder, the arrangement further comprises a circular chamber 430 comprising a tangential inlet 431 positioned along the sound axis S of piezo 411. Air blown into drug chamber 430 by blower 410 causes drug 420 to swirl around the chamber's circumference, similar to clothes in a clothes dryer. With the optional addition of internal baffles 432, the tumbling action of the drug particles 420 helps to de-agglomerate the drug. Lighter particles leave the chamber 430 through outlet 433 during patient inhalation, while the heavier particles settle back into the chamber for further tumbling.

An advantage of including a chamber 430, particularly one comprising baffles 432, is that it can be configured to control the particle size that is dispensed through the outlet 433 to ensure optimum delivery to a user. Drug particles which are of a size which are not desirable for delivery continue to be held within the chamber 430 until they break down further as required and will then be ejected. The more baffles the greater the de-agglomeration, but the baffle arrangement must also be chosen to avoid reducing the acoustic streaming effect too much.

One or more characteristics of the arrangement can be selected according to the particular drug to be delivered and the required particle size. Such characteristics include the power, frequency, size and shape of the piezo 411 and the size and shape of container 412 (all of which affect the volume and/or velocity of air driven into the chamber 430 from the blower 410), the number and/or size and/or shape of any baffles and size and/or shape and/or relative location of one or more of the main circular body of chamber 430, the inlet 431 and the outlet 433. Appropriate control of these parameters can achieve very accurate and reliable dispensing at appropriate particle size for the relevant drug to optimise delivery.

The chamber component 430 can be provided separate to the blower component 410 and can be provided as a sealed capsule prior to use either as a single component or as a blister pack. (A blister pack is an array of blisters linked together. Such an array can be a 1×n array, forming a blister strip.)

The chamber 430 can contain just a single dose of the drug 420 to be delivered, or may contain multiple doses. It can be disposable; i.e. arranged to be removed from the device after use and replaced with a fresh component 430 as necessary. Alternatively, it can be refillable through an externally accessible refill port.

If a blister pack is employed then it may comprise an array of chambers and the device may comprise a driving mechanism which drives an individual chamber within an individual blister into a position in which it is opened and in contact with the acoustic wave generator so that the acoustic wave generator can then be operated to direct acoustic waves into the chamber in the blister and on for inhalation by a user. An example of such a mechanism is described in applicant's co-pending U.S. patent application Ser. No. 62/145,923. Once the drug within the individual chamber has been dispensed the drive mechanism of the device can move the blister strip such that a subsequent blister with its chamber is placed in position for use.

Alternatively to the acoustic streaming blower 410 illustrated in FIG. 4, a synthetic jet blower 510 could be used to direct airflow into a chamber 530 as shown in FIG. 5. Synthetic jet blower 510 could be similar to the synthetic jet blower illustrated in FIG. 2, only lacking an equivalent to opening 213. It could comprise a piezo 511, a chamber 512 either directly coupled to the piezo or coupled to the piezo via a membrane or diaphragm 513, and outlet holes 514. Chamber 530 could be similar to chamber 430, wherein like reference numerals indicate like components.

Another type of blower, whose design is known from U.S. patent application publication number 2015/0071797 for use in dissipating heat generated inside a mobile electronic device or for supplying oxygen required to produce electric power in a fuel cell, could alternatively be used to de-agglomerate and/or aerosolise dry powder medicament in an inhaler device. (All variants described therein would be suitable for this application, including those described as prior art.) Such a blower has an advantageously low profile and requires very little power.

The structure of such a blower is illustrated in cross-section in FIG. 6. Blower 600 comprises a piezo 610 coupled to a diaphragm 621. A pumping chamber 620 is formed by diaphragm 621 and walls 622. Pumping chamber 620 has a substantially centrally located opening 623 in the wall 622 opposing the piezo 610.

The assembly of pumping chamber 620 and piezo 610 is mounted within housing 630 by means of connector(s) 640, which permit airflow from the piezo side of the pumping chamber to the opening side. Such connectors could be elastic. They could for example comprise a single air-permeable/perforated connector ring or a series of radial connector “spokes” distributed around the circumference of the housing.

Housing 630 comprises an outlet 631 in line with pumping chamber opening 623, optionally directing air through a nozzle 632. Housing 630 further comprises an inlet 633 to the piezo side of the pumping chamber. Inlet 633 can be provided substantially centrally as shown, i.e. in line with outlet 631, or can be provided offset from the axis joining outlet 631 and opening 623. Inlet 633 can optionally be provided with a nozzle. Multiple inlets could be provided.

When an alternating current is applied to the piezo 610, it alternately expands and contracts, causing bending vibrations in diaphragm 621, in turn causing periodic variation in the volume of the pumping chamber 620.

When the pumping chamber volume increases, air is displaced from below the piezo, some of which is pushed up into the channel 650 formed between the pumping chamber 620 and housing 630 and suctioned in to the pumping chamber through opening 623.

When the pumping chamber volume decreases, air is forced out of opening 623 through outlet 631. Air is also suctioned into the blower through inlet 633 to fill the space left below the piezo, adding further airflow through the channel 650 between the pumping chamber and housing and out of the outlet 631. Operation is enhanced by lateral movement of air within the pumping chamber, moving radially away from the centre, striking the walls 622 and returning in a phase relationship that enhances displacement out of opening 623.

The air expelled from outlet 631 is pushed out fast enough that it has sufficient momentum to avoid being sucked back in to the blower when the pumping chamber volume increases again.

Blower 600 can produce airflow strong enough to de-agglomerate and/or aerosolise dry powder medicament using very low power. For example a 1.5 lpm flow can be produced from a power input of only approximately 0.1 to 0.3 W, e.g. 0.2 W. It could for example use a 15 VPP, 25 kHz drive signal. It can also be very compact, for example having a footprint of approximately 10 to 30 mm², e.g. 20 mm², and a thickness of 1 to 3 mm, for example 1.85 mm.

To produce an even more powerful airflow, multiple blowers 600 can be arranged in series, with the outlet of one blower directed into the inlet of the next.

As shown in FIG. 7A, such a blower 710 could aim airflow directly towards drug 720, which could for example be contained in a dosing cup or open blister.

Alternatively, as shown in FIG. 7B, it could aim airflow into an inlet 731 of a chamber 730 similar to chamber 430 of FIG. 4, wherein like reference numerals indicate like components.

FIG. 7C shows a further alternative in which drug 720 is loaded into the pumping chamber itself, where it is de-agglomerated by direct vibration from the piezo through the diaphragm and expelled for dispensing (e.g. through a mouthpiece) with the airflow.

According to a further example as illustrated by FIG. 8, a blister 830 is open to a dosing chamber 810, similar to dosing chamber 210 of FIG. 2. An opening is provided opposite the blister through which airflow B from a blower (not shown) enters the chamber. Airflow B both blows dry powder medicament out of the blister 830 and pressurises dosing chamber 810 to force the powder out of outlet holes 812 into the inspiratory airflow I. Due to the pressurisation provided by the blower, no piezo or film is needed (though they could be provided in addition to the components shown). The blower could for example be similar to any of blowers 310/410, 510 or 600/710, i.e. it could be an Eckart streaming tube, a synthetic jet blower or a pumping blower.

Synthetic jetting, pumping and acoustic streaming as described above can be used to supplement patient effort in breath-actuated/tidal inhalers or to supplement/replace conventional aerosol generators in active inhalers.

When a chamber such as that shown at 420, 520 and 720 is employed, the blower need not necessarily blow directly into the tangential chamber inlet. The blower outlet and tangential inlet could instead be pneumatically coupled by a pipe. This allows for convenient placement of a blower within an inhaler. Similarly, the blower providing airflow B in FIG. 8 could blow directly into dosing chamber 810, or could be pneumatically coupled thereto by a pipe.

Other embodiments will be apparent to those skilled in the art from consideration of the specification and practice of the embodiments disclosed herein. It is intended that the specification and examples be considered as exemplary only.

In addition, where this application has listed the steps of a method or procedure in a specific order, it could be possible, or even expedient in certain circumstances, to change the order in which some steps are performed, and it is intended that the particular steps of the method or procedure claims set forth herein not be construed as being order-specific unless such order specificity is expressly stated in the claim. That is, the operations/steps may be performed in any order, unless otherwise specified, and embodiments may include additional or fewer operations/steps than those disclosed herein. It is further contemplated that executing or performing a particular operation/step before, contemporaneously with, or after another operation is in accordance with the described embodiments.

1. A de-agglomerator for a dry powder inhaler, said de-agglomerator comprising:
a medicament chamber for housing dry powder medication, said medicament chamber comprising an inlet and an outlet and having a cross-section with a substantially circular inner perimeter;
a conduit in pneumatic communication with said inlet; and
a blower arranged to direct airflow at said dry powder medication through said conduit such that the medicament swirls around at least a part of said perimeter.

2. The de-agglomerator of claim 1, wherein the conduit is tangential to the perimeter.

3. The de-agglomerator of claim 1, wherein the blower comprises a piezoelectric transducer, an acoustic streaming blower, a synthetic jet blower or a pumping blower.

4. The de-agglomerator of claim 1, wherein the medicament chamber comprises one or more internal baffles.

5. (canceled)

6. The de-agglomerator of claim 3, wherein said blower comprises a tube closed at one end by said transducer, an open end of said tube being in pneumatic communication with the conduit.

7. The de-agglomerator of claim 6, wherein said transducer is, or is coupled to, a focussing dish, concave with respect to the airflow direction.

8. (canceled)

9. The de-agglomerator of claim 3, wherein said blower comprises a pumping chamber coupled to said transducer and having at least one outlet hole in pneumatic communication with the conduit.

10. The de-agglomerator of claim 9, wherein said outlet hole is in a wall of said pumping chamber opposing the transducer.

11. The de-agglomerator of claim 9, wherein the transducer is coupled to the pumping chamber through a diaphragm.

12. The de-agglomerator of claim 10, wherein the outlet hole is in line with the transducer axis.

13. The de-agglomerator of claim 11, wherein the pumping chamber is surrounded by a housing, said housing:
comprising a housing inlet in a wall facing the transducer;
comprising a housing outlet in a wall facing the outlet hole and in line with the outlet hole; and
being spaced from the pumping chamber by an airflow channel in pneumatic communication with the housing inlet and the housing outlet.

14. A method of de-agglomerating dry powder medicament in an inhaler, said method comprising, using a blower, directing airflow at said medicament through a conduit such that the medicament swirls around at least a part of a substantially circular inner perimeter of a medicament chamber housing the medicament, said medicament chamber comprising an inlet in pneumatic communication with said conduit and an outlet.

15. A de-agglomerator for a dry powder inhaler, said de-agglomerator comprising:
a piezoelectric transducer;
a pumping chamber:
coupled to said transducer through a diaphragm, and
having a outlet hole:
in a wall opposing the transducer,
in line with the transducer axis, and
arranged to be in pneumatic communication with a flow channel of said inhaler through which a user can inhale; and
a housing surrounding said pumping chamber, said housing:
comprising a housing inlet in a wall facing the transducer;
comprising a housing outlet in a wall facing said outlet hole and in line with the outlet hole; and
being spaced from the pumping chamber by an airflow channel in pneumatic communication with the housing inlet and the housing outlet.

16. The de-agglomerator of claim 15, wherein said pumping chamber is arranged for housing dry powder medicament.

17. The de-agglomerator of claim 15, further comprising a dosing chamber for housing dry powder medication, said dosing chamber comprising:
an opening configured to receive an open dry powder medicament blister such that dry powder medicament can pass into the dosing chamber from said blister;
an outlet in pneumatic communication with said flow channel; and
an inlet in pneumatic communication with said outlet hole, configured to direct airflow from the outlet hole at said opening.

18. The de-agglomerator of claim 15, further comprising:
a medicament chamber for housing dry powder medication, said medicament chamber comprising an inlet and an outlet in pneumatic communication with said flow channel and having a cross-section with a substantially circular inner perimeter; and
a conduit in pneumatic communication with said inlet;
wherein the outlet hole is arranged to direct airflow at said dry powder medication through said conduit such that the medicament swirls around at least a part of said perimeter.

19. The de-agglomerator of claim 18, wherein the conduit is tangential to the perimeter.

20. The de-agglomerator of claim 18, wherein the medicament chamber comprises one or more internal baffles.

21. A capsule comprising a medicament chamber and arranged for use in the de-agglomerator of claim 1.

22. A blister pack comprising an array of the capsules of claim 21.

23. A method of de-agglomerating dry powder medicament in an inhaler, said method comprising:
providing a pumping chamber with said dry powder medicament; and
supplying an alternating current to a piezoelectric transducer coupled to said pumping chamber through a diaphragm such that the volume of the pumping chamber varies with the supplied voltage, the pumping chamber:
having a outlet hole:
in a wall opposing the transducer,
in line with the transducer axis, and
arranged to be in pneumatic communication with a flow channel of said inhaler through which a user can inhale,
the pumping chamber being surrounded by a housing, said housing:
comprising a housing inlet in a wall facing the transducer;
comprising a housing outlet in a wall facing said outlet hole and in line with the outlet hole; and
being spaced from the pumping chamber by an airflow channel in pneumatic communication with the housing inlet and the housing outlet.

24. A method of de-agglomerating dry powder medicament in an inhaler, said method comprising:
providing a medicament chamber with said dry powder medicament, said medicament chamber:
comprising an inlet and an outlet,
being in pneumatic communication, via said outlet, with a flow channel of said inhaler through which a user can inhale, and
having a cross-section with a substantially circular inner perimeter; and
supplying an alternating current to a piezoelectric transducer coupled to a pumping chamber through a diaphragm such that the volume of the pumping chamber varies with the supplied voltage, the pumping chamber:
having a outlet hole:
in a wall opposing the transducer,
in line with the transducer axis, and
arranged to direct airflow at said dry powder medication through a conduit in pneumatic communication with said inlet such that the medicament swirls around at least a part of said perimeter,
the pumping chamber being surrounded by a housing, said housing:
comprising a housing inlet in a wall facing the transducer;
comprising a housing outlet in a wall facing said outlet hole and in line with the outlet hole; and
being spaced from the pumping chamber by an airflow channel in pneumatic communication with the housing inlet and the housing outlet.

25. An inhaler comprising the de-agglomerator of claim 1.

26. A capsule comprising a medicament chamber and arranged for use in the de-agglomerator of claim 15.

27. An inhaler comprising the de-agglomerator of claim 15.
